(19) United States
(12) Patent Application Publication
(10) Pub. No.: US 2006/0079763 A1
(43) Pub. Date: Apr. 13, 2006

(54) Backprojected patient multimedia display

(75) Inventors: Andrew G. Jeung, Mountain View, CA (US); Hassan Mostafavi, Los Altos, CA (US)

Correspondence Address: VARIAN MEDICAL SYSTEMS TECHNOLOGIES, INC.;c/o BINGHAM MCCUTCHEN LLP, THREE EMBARCADERO CENTER, SAN FRANCISCO, CA, 94111-4067, US

(73) Assignee: Varian Medical Systems Technologies, Inc., Palo Alto, CA (US)

(21) Appl. No.: 11/217,789

(22) Filed: Aug. 30, 2005

Related U.S. Application Data
(63) Continuation-in-part of application No. 10/956,199, filed on Sep. 30, 2004.

(51) Int. Cl. A61B 5/05

(52) U.S. Cl. 600/428

(57) ABSTRACT

An apparatus for prompting a patient includes a structure configured to be mounted to a patient support, a screen coupled to the structure, and a projector located at a distance away from the screen. A method of prompting a patient that is being supported on a patient support includes adjusting a position of a screen relative to a projector, the screen having a surface, placing the screen in front of the patient such that the patient can see the surface, and providing an image on the screen using the projector.

### RELATED APPLICATION DATA

This application is a continuation-in-part of U.S. patent application Ser. No. 10/956,199, filed Sep. 30, 2004, entitled “Patient Multimedia Display”, the entire disclosure of which is expressly incorporated by reference herein.

This application is related to U.S. patent application Ser. No. 10/957,009, filed Sep. 30, 2004, entitled “Patient Visual Instruction Techniques For Synchronizing Breathing With a Medical Procedure”, the entire disclosure of which is expressly incorporated by reference herein.

### BACKGROUND OF THE INVENTION

1. Field of the Invention

This invention relates generally to systems and methods for prompting patient, and more specifically, to systems and methods for prompting patient to control patient movement.

2. Background of the Invention

Computed tomography is an imaging technique that has been widely used in the medical field. In a procedure for computed tomography, an x-ray source and a detector apparatus are positioned on opposite sides of a portion of a patient under examination. The x-ray source generates and directs a x-ray beam towards the patient, while the detector apparatus measures the x-ray absorption at a plurality of transmission paths defined by the x-ray beam during the process. The detector apparatus produces a voltage proportional to the intensity of incident x-rays, and the voltage is read and digitized for subsequent processing in a computer. By taking thousands of readings from multiple angles around the patient, relatively massive amounts of data are thus accumulated. The accumulated data are then analyzed and processed for reconstruction of a matrix (visual or otherwise), which constitutes a depiction of a density function of the bodily section being examined. By considering one or more of such sections, a skilled diagnostician can often diagnose various bodily ailments such as tumors, blood clots, etc.

Computed tomography has found its principal application to examination of bodily structures or the like which are in a relatively stationary condition. However, currently available computed tomographic apparatus may not be able to generate tomographic images with sufficient quality or accuracy due to physiological movement of a patient. For example, beating of a human heart and breathing have been known to cause degradation of quality in CT images.

Degradation of quality of CT images due to patient's breathing is more difficult to address than that associated with heart motion. Patients' breathing poses a unique problem to CT imaging that is different from heart motion. This is because the pattern and the period of a patient's breathing cycle is generally less consistent when compared to those of the patient's cardiac cycle. As such, while a particular phase of a cardiac cycle may be predicted with sufficient accuracy, a particular phase of a breathing cycle may not be as easily predicted or determined. Furthermore, there has been an increased desire to visualize organ motion by viewing a sequence of CT images as a movie sequence. However, collecting a large quantity of CT image data sufficient for forming a video while considering breathing motion is difficult to perform and may take a much longer time.

For the foregoing, it would be desirable to prompt a patient to control the patient's breathing as CT image data are collected. The controlling can be in the form of 1) issuing periodic visual and audio commands to regularize the respiration motion so that a CT sequence can be formed as a function of the phase of breathing, or 2) using visual and audio commands to prompt the patient to hold breath at specific times and periods as required by the image acquisition process. Although visual signals have been used to prompt patients, use of visual prompting signals have been avoided in radiation procedures. This is because most image devices, such as a computer screen, is too large to fit within the bore of a CT machine. Even for those image devices that could fit within the bore of a CT machine, the image device will take up a lot of space within the bore. This may cause a patient who is confined within a gantry opening to feel uncomfortable—especially if the patient is claustrophobic. Also, electronics of an image device may interfere with a radiation field generated during a CT procedure.

### SUMMARY

In accordance with some embodiments, an apparatus for prompting a patient includes a structure configured to be mounted to a patient support, a screen coupled to the structure, and a projector located at a distance away from the screen.

In accordance with other embodiments, a method of prompting a patient that is being supported on a patient support includes adjusting a position of a screen relative to a projector, the screen having a surface, placing the screen in front of the patient such that the patient can see the surface, and providing an image on the screen using the projector.

Other aspects and features will be evident from reading the following detailed description of the preferred embodiments, which are intended to illustrate, not limit, the invention.

### BRIEF DESCRIPTION OF THE DRAWINGS

The drawings illustrate the design and utility of various embodiments, in which similar elements are referred to by common reference numerals. In order to better appreciate how advantages and objects of the embodiments are obtained, a more particular description of the embodiments will be illustrated in the accompanying drawings. As such, these drawings depict only embodiments and are not therefore to be considered limiting of its scope.

FIG. 1 illustrates a computed tomography system having a patient prompting device in accordance with some embodiments;

FIG. 2 illustrates a perspective view of the patient prompting device of FIG. 1;

FIG. 3 illustrates a side view of the patient prompting device of FIG. 1, showing the patient prompting device being used to prompt a patient;

FIG. 4 illustrates a perspective view of a patient prompting device in accordance with other embodiments;

FIG. 5 illustrates a side view of the patient prompting device of FIG. 4, showing the patient prompting device being used to prompt a patient;

FIG. 6 illustrates a perspective view of a patient prompting device in accordance with other embodiments;

FIG. 7 illustrates a perspective view of a patient prompting device in accordance with other embodiments;

FIG. 8 illustrates a perspective view of a patient prompting device having a focusing device in accordance with other embodiments; and

FIG. 9 illustrates a perspective view of a patient prompting device in accordance with other embodiments.

### DETAILED DESCRIPTION OF THE EMBODIMENTS

Various embodiments are described hereinafter with reference to the figures. It should be noted that the figures are not drawn to scale and elements of similar structures or functions are represented by like reference numerals throughout the figures. It should also be noted that the figures are only intended to facilitate the description of specific embodiments. They are not intended as an exhaustive description of the invention or as a limitation on the scope of the invention. In addition, an aspect described in conjunction with a particular embodiment is not necessarily limited to that embodiment and can be practiced in any other embodiments.

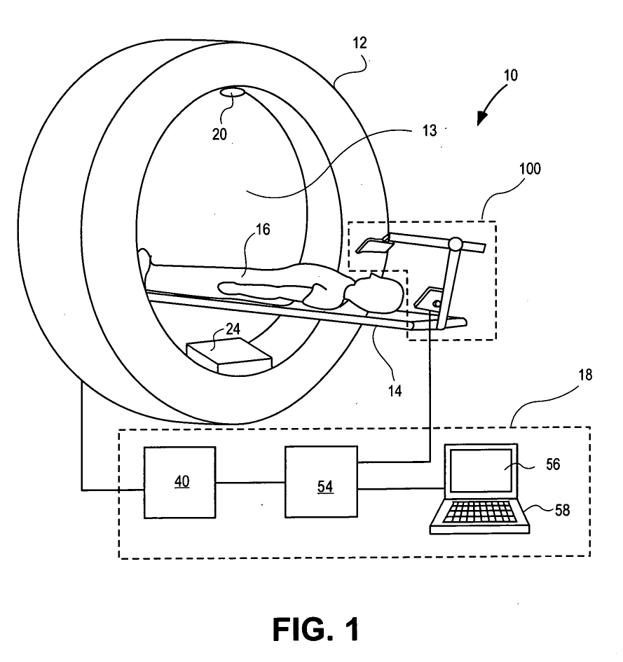

Referring now to the drawings, in which similar or corresponding parts are identified with the same reference numeral, FIG. 1 illustrates a computed tomography image acquisition system 10, in which embodiments of the present invention can be employed. The system 10 includes a gantry 12 having an opening (or bore) 13, a patient support 14 for supporting a patient 16, and a control system 18 for controlling an operation of the gantry 12. The system 10 also includes an x-ray source 20 that projects a beam of x-rays towards a detector 24 on an opposite side of the gantry 12 while the patient 16 is positioned at least partially between the x-ray source 20 and the detector 24. The detector 24 has a plurality of sensor elements configured for sensing a x-ray that passes through the patient 16. Each sensor element generates an electrical signal representative of an intensity of the x-ray beam as it passes through the patient 16.

In the illustrated embodiment, the control system 18 includes a processor 54, such as a computer processor, coupled to a patient prompting device 100 and to a gantry rotation control 40. The control system 18 may also include a monitor 56 for displaying data and an input device 58, such as a keyboard or a mouse, for inputting data. During a scan to acquire x-ray projection data (i.e., CT image data), the gantry 12 rotates about the patient 16. The rotation of the gantry 12 and the operation of the x-ray source 20 are controlled by the gantry rotation control 40, which provides power and timing signals to the x-ray source 20 and controls a rotational speed and position of the gantry 12 based on signals received from the processor 54. Although the control 40 is shown as a separate component from the gantry 12 and the processor 54, in alternative embodiments, the control 40 can be a part of the gantry 12 or the processor 54. The processor 54 is configured to send prompting signals to the patient prompting device 100 in a prescribed manner (e.g., in synchronization with a rotation of the gantry 12).

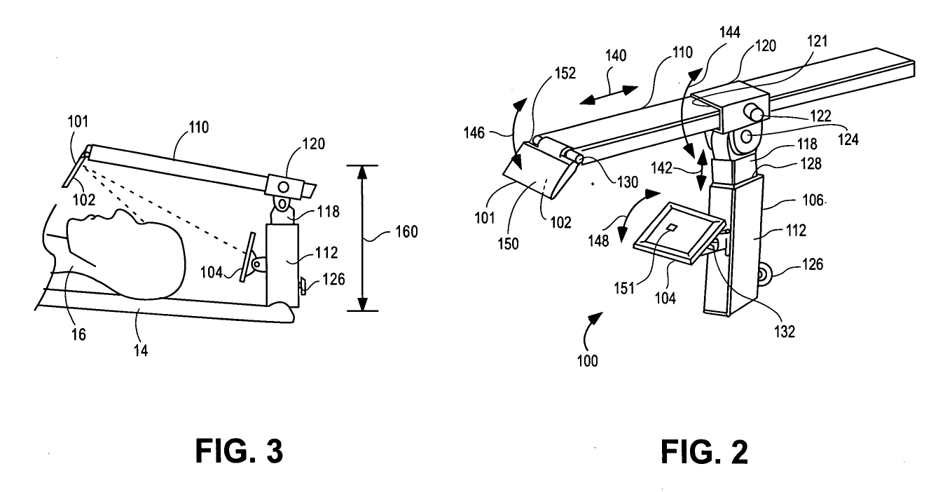

The patient prompting device 100 is configured to provide visual signals to the patient 16 during a procedure, thereby instructing the patient 16 to perform certain task(s). FIG. 2 shows the patient prompting device 100 in accordance with some embodiments of the invention. The patient prompting device 100 includes a screen 101 having a surface 102 between a first side 150 and a second side 152, an image source 104, and a structure 106 to which the screen 101 and the image source 104 are coupled.

In some embodiments, the screen 101 is made from a non-metallic material and does not include circuitry for preventing interference with a radiation field. In other embodiments, the screen 101 may have insubstantial circuitry, which is defined herein as circuitry that does not substantially interfere with the imaging or other treatment procedure being performed. For example, the screen 101 may comprise simple circuits, such as an LED and associated components to provide signals to the patient, that indicate status, etc, such as an LED indicating that the imaging system is on. Depending upon the imaging modality and the ability to tolerate image imperfections, such insubstantial circuitry may be more complex, but still comprise substantially less circuitry than a screen such as an LCD screen where the image is formed entirely or substantially from the LCD imaging circuitry. For example, such insubstantial circuitry may form a part of an image or form an image that is overlaid on a projected image.

In other embodiments, the screen 101 can be any object as long as it provides a surface. In the illustrated embodiments, the surface 102 is a mirror surface, and the image source 104 includes a flat panel screen (or a monitor screen). During use, the image source 104 receives image data from the processor 54 and displays an image 151 in response thereto. The image 151 is reflected by the mirror surface 102, and the patient 16 can see the reflected image 151 by looking towards the mirror surface 102 (FIG. 3). The image 151 displayed on the image source 104 is in reverse (or flipped) such that the patient 16 can see a reflection of the image 151 in a non-reverse (or intended) manner using the mirror surface 102. In the illustrated embodiments, the image 151 provides visual signal to control the patient's breathing (e.g., by instructing the patient 16 to hold breath, to inhale, and/or to exhale). One application is to synchronize the patient breathing to a process being performed by a treatment or imaging device. For example, the patient breathing can be synchronized with a motion of the gantry 12 as the gantry 12 rotates around the patient 16 to collect image data, thereby ensuring that image data that correspond to a prescribed phase of a breathing cycle are obtained. However, in other embodiments, the image 151 can be configured to instruct the patient 16 to perform other task(s), such as, to relax, to move an arm or a leg, to respond to a question, etc.

The above described configuration of the patient prompting device 100 is advantageous because it keeps the electronics of the image source 104 away from a radiation field generated by the x-ray source 20, thereby preventing the electronics of the image source 104 from interfering a CT procedure. Also, it minimizes damage to the image source 104 due to X-ray radiation in a treatment machine. In addition, such configuration provides comfortable viewing of the image 151 because the patient 16 does not need to focus directly onto the image source 104. Also, patients that are far sighted will not need to use reading glasses because reflection through mirror increases a length of the viewing path. Further, the low profile 160 of the prompting device 100 allows the device 100 itself to be placed inside the bore 13 of the CT gantry 12 (or other machines, such as a PET scanner).

The position of the screen 101 can be adjusted relative to the image source 104 to accommodate different patients and/or different applications. In the illustrated embodiments, the structure 106 includes a first arm 110 for carrying the screen 101, a second arm 112 for carrying the image source 104, and connecting members 120, 118 for coupling the first arm 110 to the second arm 112. Particularly, the screen 101 is rotatably coupled to an end of the first arm 110 via a shaft 130, thereby allowing the screen 101 to rotate (as indicated by the arrows 146) relative to the first arm 110. Coupling the screen 101 to the first arm 110 using the second side 152 (i.e., instead of the first side 150) of the screen 101 is advantageous because it allows the first arm 110 to be spaced further from a patient's head, thereby providing more level of comfort to the patient 16. Similarly, the image source 104 is rotatably coupled to the second arm 112 via a shaft 132, thereby allowing the image source 104 to rotate (as indicated by the arrows 148) relative to the second arm 112. The connecting member 120 includes a slot 121 through which the first arm 110 can be inserted, and a knob 122 for securing the first arm 110 relative to the connecting member 120. Such configuration allows the first arm 110 to be translated (in the directions 140), thereby adjusting a position of the screen 101. The connecting member 120 is rotatably secured to the connecting member 118 via a shaft 124, thereby allowing the first arm 110 to rotate relative to the second arm 112 (as indicated by the arrows 144). The connecting member 118 is sized to fit within a lumen 128 of the second arm 112, and is slidable relative to the second arm 112 (as indicated by arrow 142) for adjusting a height of the screen 101. In the illustrated embodiments, the connecting member 118 and the second arm 112 each has a non-circular cross section. However, in alternative embodiments, the connecting member 118 and the second arm 112 can each have a circular cross section, in which case, the connecting member 118 can be rotated about its axis relative to the second arm 112 to place the screen 101 at a desired position. A knob 126 is provided for securing the connecting member 118 relative to the second arm 112 after the connecting member 118 has been desirably positioned. In some embodiments, one or more of the components of the structure 106, such as the first arm 110, the surface, and the joining mechanism, can all be made from a non-metallic material, such as carbon graphite or a polymer, to minimize interference with a radiation field.

The above described structure 106 is advantageous because it allows a position of the screen 101 to be adjusted in multiple directions. However, it should be noted that the structure 106 should not be limited to that described previously, and that the structure 106 can also have other shapes and configurations. For example, in alternative embodiments, the structure 106 can have more or less than two arms (e.g., arms 110, 112). Also, in other embodiments, if two or more arms are provided, one of the arms of the structure 106 can be configured to be moveable or non-moveable relative to another arm, and an orientation of one of the arms relative to another of the arms can be different from that described previously. In addition, in other embodiments, instead of, or in addition to, any of the type of movement characteristics of the screen 101 described previously, the structure 106 can have different number of arms connected by different types of connections to provide desired movement characteristic(s) for the screen 101 (relative to the image source 104 or to the patient support 14). Further, instead of arm(s) or elongated elements, the structure 106 carrying the screen 101 can include other structural elements, such as a block, a plate, a mechanical component, etc. As such, the structure 106 can be any object as long as it is capable of holding the screen 101 at a position relative to the image source 104.

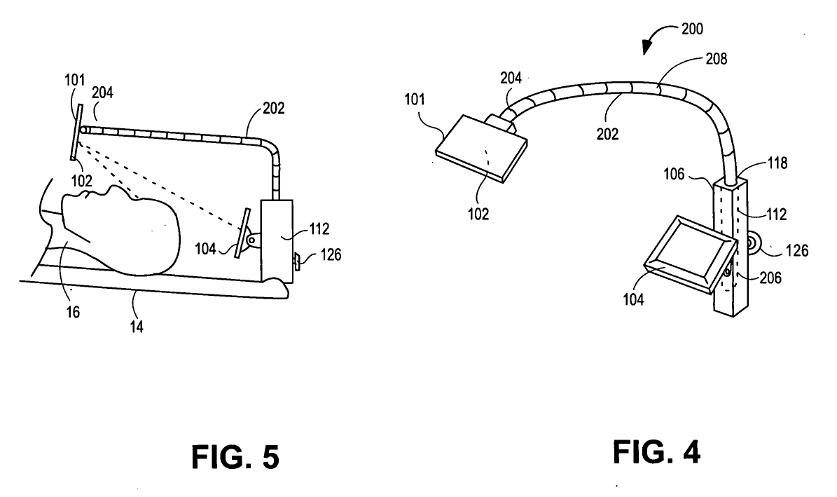

FIG. 4 illustrates another patient prompting device 200 in accordance with other embodiments of the invention. Similar to the patient prompting device 100, the patient prompting device 200 includes the screen 101 having the surface 102, and the image source 104. However, unlike the patient prompting device 100, the structure 106 of the patient prompting device 200 does not include the first arm 110. Instead, the structure 106 includes a bellow 202 for holding the screen 101 at a desired position relative to the image source 104. The bellow 202 includes a first end 204 to which the screen 101 is secured, and a second end 206 that is inserted into the lumen 118 of the arm 112. The bellow 202 includes a plurality of segments 208 that can be positioned relative to an adjacent segment 208, thereby allowing the bellow 202 to be bent to a desired profile during use. Such connection is also known as a “goose neck” joint. During use, the image source 104 receives image data from the processor 54 and displays an image 151 in response thereto. The image 151 is reflected by the mirror surface 102, and the patient can see a reflection of the image 151 by looking at the mirror surface 102 (FIG. 5). In alternative embodiments, the structure 106 can include a second bellow for connecting the image source 102 to the arm 112, to the first bellow 208, or to the patient support 14 (in which case, the arm 112 is not required). Also, in other embodiments, instead of using a below, the structure 106 can include another type of bendable element, such as an elastic polymer shaft.

Although the patient prompting device has been described as having a mirror surface, the scope of the invention should not be so limited. In other embodiments, the patient prompting device can include a non-mirror (e.g., a non-reflective) surface. In such cases, instead of the image source 104 being a flat panel or a screen, the image source 104 includes an image projector that projects image onto the surface 102. Also, in other embodiments, the image source 104 can include fiber optics for transmitting image signals to a viewing surface. In such case, the screen 101 can be a component of a glasses or goggles, with the viewing surface 102 being an inside face of the glasses or goggles. Other types of image source can also be used in alternative embodiments.

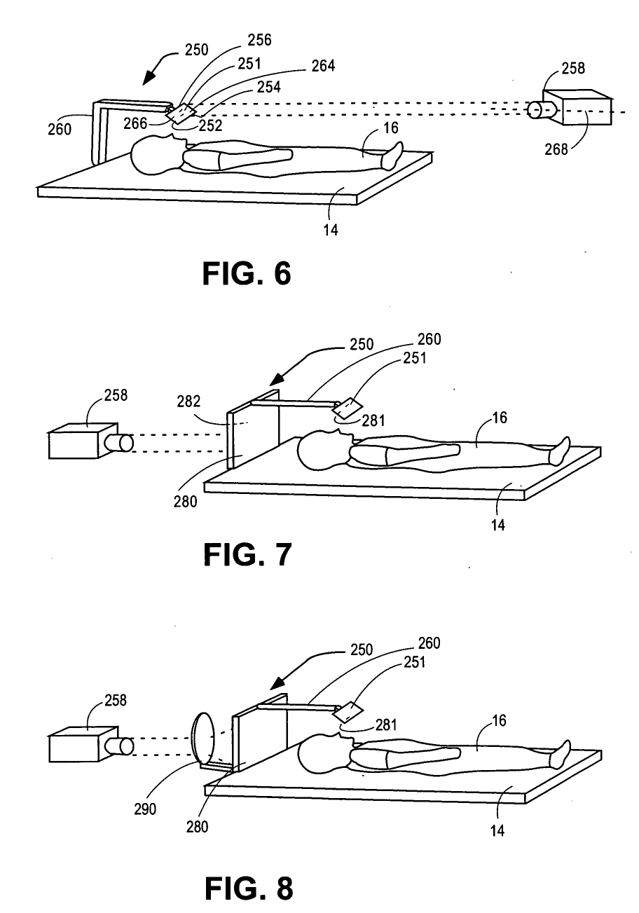

FIG. 6 illustrates a patient prompting device 250 in accordance with other embodiments. The patient prompting device 250 includes a screen 251 having a surface 252 between a first side 254 and a second side 256, an image source 258, and a structure 260 to which the screen 251 and the image source 258 are coupled. The structure 260 can have a configuration that is similar to the structure 106 of FIG. 2 or FIG. 4. In other embodiments, the structure 260 can have other configurations as long as it can secure the screen 251 relative to the patient support 14. In some embodiments, the screen 251 is a non-electronic screen (i.e., does not include circuitry), and made from a non-metallic material, for preventing interference with a radiation field. In other embodiments, the screen 251 includes an insubstantial amount of circuitry (or less) such that the screen 251 does not substantially interfere with an operation of the system 10. For example, in some embodiments, the screen 251 does not include imaging circuitry for a LCD device. In other embodiments, the screen 251 can be any object as long as it provides a surface. In the illustrated embodiments, the screen 251 is a projection screen, and the image source 258 is a projector for back-projecting image(s) to the screen 251.

During use, the image source 258 receives image data from the processor 54 and displays an image in response thereto. The image is projected onto one side 264 of the screen 251, and the patient 16 can see the image by looking towards an opposite side 266 of the screen 251. In the illustrated embodiments, the image provides visual signal to control the patient's breathing (e.g., by instructing the patient 16 to hold breath, to inhale, and/or to exhale). One application is to synchronize the patient breathing to a process being performed by a treatment or imaging device. For example, the patient breathing can be synchronized with a motion of the gantry 12 as the gantry 12 rotates around the patient 16 to collect image data, thereby ensuring that image data that correspond to a prescribed phase of a breathing cycle are obtained. However, in other embodiments, the image can be configured to instruct the patient 16 to perform other task(s), such as, to relax, to move an arm or a leg, to respond to a question, etc.

In the illustrated embodiments, the image source 258 is located at a distance away from the patient 16. Such configuration is advantageous because the electrical components of the image source 258 will not interfere with a radiation field generated during a CT procedure. As shown in the figure, the image source 258 is located adjacent to a bottom of the patient support 14 (where the legs of the patient 16 are supported). Because the screen 251 is tilted such that its surface is non-perpendicular to an axis 268 of the image source 258, a portion of the projected image near the second side 256 will appear larger than another portion of the projected image near the first side 254. In some embodiments, the processor 54 can be configured (e.g., through software/programming) to provide altered image to compensate for a tilting of the screen 251, thereby allowing the patient 16 to view image that appears normal on the screen 251. In other embodiments, the image source 258 can be located at other positions relative to the screen 251. For example, the image source 258 can be positioned such that its axis 268 is substantially perpendicular (e.g., 90°±10°) to the surface of the screen 251. In such cases, the image as it appears on the screen 251 will appear normal to the patient 16.

FIG. 7 illustrates a variation of the embodiments of FIG. 6, in which the image source 258 is located adjacent to a top of the patient support 14 (where the head of the patient 16 is supported). In such cases, the patient prompting device 250 further includes a projection screen 280, and the screen 251 includes a mirror surface 281. During use, the image source 258 receives image data from the processor 54 and displays an image in response thereto. The image is projected onto one side 282 of the projection screen 280, and the patient 16 can see the image by looking towards the mirror surface 281 of the screen 251. As similarly discussed herein, in some embodiments, the processor 54 can optionally be configured (e.g., through software/programming) to provide altered image to compensate for a tilting of the screen 251, thereby allowing the patient 16 to view image that appears normal on the screen 251.

In the embodiments of FIGS. 6 and 7, the image source 258 is secured to the patient support 14 (e.g., through a mechanical arm or connection). Such configuration is advantageous in that it allows the image source 258 to move together with the patient support 14 as the patient support 14 is being positioned. In other embodiments, the image source 258 can be secured to other objects, such as a wall, a ceiling, a CT machine or any of other objects adjacent to the patient support 14. In such cases, the processor 54 and/or the image source 258 can be configured to adjust a zooming and a focusing of the image(s) as the patient support 14 is being positioned relative to the image source 258.

FIG. 8 illustrates another variation of the patient prompting device 250 in which the patient prompting device 250 further includes a focusing device 290 located between the image source 258 and the projection screen 280. In some embodiments, the focusing device 290 may include a set of lenses in a telescopic arrangement such as those found in modern cameras, or a two-dimensional lens array that is known in the art. In such cases, the image source 258 is not secured to the patient support 14, and is configured to focus image(s) at a far distance (e.g., at a distance that is between 5 feet and infinity, and more preferably, more than 20 feet, away from the image source 258). The focusing device 290 is secured relative to the projection screen 280 such that it will move together with the projection screen 280 as the patient support 14 is being positioned. The focusing device 290 will focus image(s) correctly onto the projection screen 280 regardless of the distance between the image source 258 and the projection screen 280.

In other embodiments, the patient prompting device 100, 200, or 250 can further include a connection mechanism for connecting the structure 106 to the patient support 14. The connection mechanism can include, for examples, a clamp, a screw knob, or a pull-and-release type knob. In some cases, the connection mechanism can include one or more members connected to the structure 106 for mating with respective receiving members on the patient support 14. In other embodiments, the patient prompting device 100 or 200 can further include the patient support 14, in which case, the prompting device 100 or 200 can be fixedly secured to the patient support 14 (e.g., via a weld, a bolt, or a screw), or be detachably secured to the patient support 14.

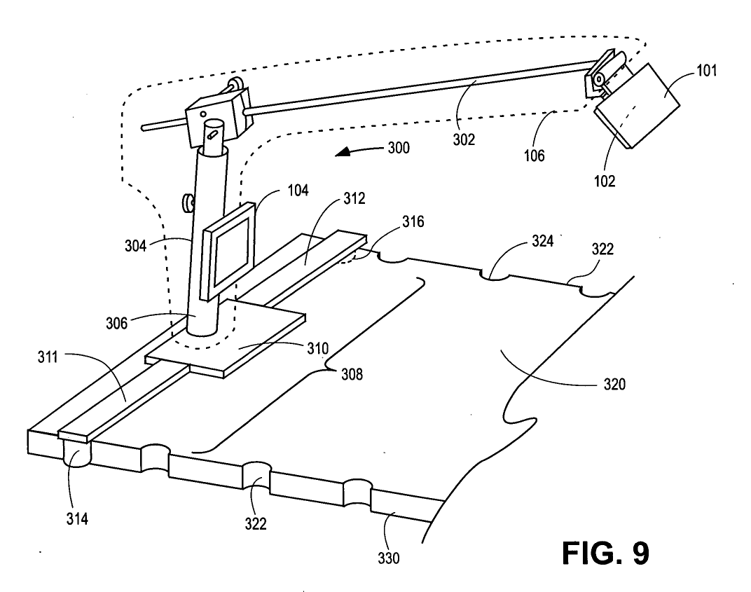

FIG. 9 illustrates a patient prompting device 300 that is configured to be detachably secured to a patient support 320. The patient prompting device 300 includes the screen 101, the image source 104, and the structure 106, and is similar to the patient prompting device 100 described previously. The structure 106 includes a rod 302 (first arm) coupled to a support 304 (second arm), with the support 304 having an end 306 that is attached to a securing mechanism 308. The securing mechanism 308 includes a plate 310, members 311, 312 extending from the plate 310, and securing elements 314, 316 located at respective ends of the members 311, 312. The securing elements 314, 316 can be, for example, circular disks, or other types of fastening members. In the illustrated embodiments, the patient support 304 includes a plurality of recesses 322 on a first edge 330, and a plurality of recesses 324 on a second edge 332. The securing elements 314, 316 are configured to mate with the one of the recesses 322 and one of the recesses 324, respectively, on both sides of the patient support 320. The plurality of recesses 322, 324 allow a position of the patient prompting device 300 be adjusted relative to the support 320. The securing mechanism 308 and the patient support 320, and variations thereof, have been described in U.S. Pat. No. 5,806,116, the entire disclosure of which is expressly incorporated by reference herein.

Also, in some embodiments, the patient prompting device 100 can include a processor, such as the processor 54, for processing image signals/data. Further, in other embodiments, the patient prompting device 100 or 200 can further include one or more speakers for providing audio signal to the patient 16 in addition to the visual signal 50. For example, the speaker(s) can be integrated speaker(s) that is part of the image source 104. Alternatively, the speaker(s) can be separate speaker(s) that is secured to the structure 106 or to the patient support 14. In addition, in other embodiments, the image source 104 can be configured to receive audio and/or video signals by one or more wireless connections. In such cases, the image source 104 includes its own power source and a wireless receiver for receiving signals from a transmitter.

Although embodiments of the patient prompting device have been described as being used with the computed tomography image acquisition system 10, in alternative embodiments, any of the embodiments of the patient prompting device described herein can be used to control patient motion in other types of radiation process. For examples, instead of a CT procedure, any of the above described patient prompting devices can be used in a laminar tomography procedure, a MRI procedure, a PET procedure, or other imaging procedures. Also, in other embodiments, instead of using the patient prompting device in image acquisition procedures, any of the above described patient prompting devices can be used in a treatment procedure, such as a radiation treatment procedure that requires a synchronization of a patient's movement to a treatment machine. In addition, in further embodiments, any of the embodiments of the patient prompting device described herein can be used in different applications, which may or may not require use of a radiation machine.

Although particular embodiments have been shown and described, it will be understood that they are not intended to limit the present inventions, and it will be obvious to those skilled in the art that various changes and modifications may be made without departing from the spirit and scope of the present inventions. For example, in other embodiments, instead of securing the image source 104 to the structure 106, the image source 104 can be secured to the patient support 14, or to another structure that is coupled to the patient support 14. In such cases, the patient prompting device 100 or 200 does not include the image source 104. Also, in other embodiments, the image source 104 is not limited to a single flat panel screen, a single monitor screen, or a single projector, and can include multiple image-providing devices (e.g., multiple flat panel screens, monitor screens, or projectors). For example, multiple image-providing devices can be used to provide 2-dimensional projection or holographic. The specification and drawings are, accordingly, to be regarded in an illustrative rather than restrictive sense. The present inventions are intended to cover alternatives, modifications, and equivalents, which may be included within the spirit and scope of the present inventions as defined by the claims.

What is claimed:

1. An apparatus for prompting a patient, comprising:
a structure configured to be mounted to a patient support;
a screen coupled to the structure; and
a projector located at a distance away from the screen.

2. The apparatus of claim 1, wherein the projector is configured for providing backprojected images.

3. The apparatus of claim 1, wherein the patient support has a first portion for supporting a patient's head and a second portion for supporting a patient's legs, and the projector is located closer to the first portion than the second portion.

4. The apparatus of claim 1, wherein the patient support has a first portion for supporting a patient's head and a second portion for supporting a patient's legs, and the projector is located closer to the second portion than the first portion.

5. The apparatus of claim 1, wherein the screen comprises a mirror surface.

6. The apparatus of claim 1, wherein the screen comprises a non-mirror surface.

7. The apparatus of claim 1, wherein the projector is secured to the patient support.

8. The apparatus of claim 1, wherein the patient support is moveable relative to the projector.

9. The apparatus of claim 1, further comprising a focusing device for focusing an image provided by the projector.

10. The apparatus of claim 1, wherein the structure comprises a first arm and a second arm movable relative to the first arm, wherein the screen is secured to the second arm, and the first arm is configured to be secured to the patient support.

11. The apparatus of claim 1, further comprising a processor coupled to the projector.

12. The apparatus of claim 11, wherein the processor is configured to cause the projector to display the image for patient prompting.

13. The apparatus of claim 12, wherein the image comprises a visual signal for prompting the patient to control the patient's breathing.

14. The apparatus of claim 1, wherein the screen is translatable relative to the image source.

15. The apparatus of claim 1, wherein the screen is rotatable relative to the image source.

16. The apparatus of claim 1, wherein the screen includes no more than insubstantial circuitry.

17. A method of prompting a patient that is being supported on a patient support, comprising:
positioning a screen relative to a projector, the screen having a surface;
placing the screen in front of the patient such that the patient can see the surface; and
providing an image on the screen using the projector.

18. The method of claim 17, wherein the surface comprises a mirror surface.

19. The method of claim 17, wherein the surface does not comprise a mirror surface.

20. The method of claim 17, wherein the image comprises a visual signal for prompting the patient to control the patient's breathing.

21. The method of claim 17, wherein the providing comprises projecting an image directly onto the screen.

22. The method of claim 17, wherein the providing comprises projecting an image onto a projection screen that is located adjacent to the screen.

23. The method of claim 17, wherein the providing comprises focusing an image at a distance that is at least 20 feet away from the projector.

24. The method of claim 17, wherein the screen includes no more than insubstantial circuitry.

25. The method of claim 17, wherein the steps of positioning the screen and placing the screen in front of the patient are performed substantially simultaneously.
